(19) United States
(12) Patent Application Publication
(10) Pub. No.: US 2025/0152344 A1
(43) Pub. Date: May 15, 2025

(54) APPARATUS AND METHOD FOR REPLACING A DISEASED CARDIAC VALVE

(71) Applicant: EDWARDS LIFESCIENCES CORPORATION, Irvine, CA (US)

(72) Inventors: Jose Luis Navia, Shaker Heights, OH (US); Ji-Feng Chen, Lakewood, OH (US); Qun Zhou, Lakewood, OH (US)

(21) Appl. No.: 19/024,370

(22) Filed: Jan. 16, 2025

Related U.S. Application Data
(63) Continuation of application No. 17/067,580, filed on Oct. 9, 2020.
Application No. 17/067,580 is a continuation of application No. 12/769,593, filed on Apr. 28, 2010, now abandoned.
(60) Provisional application No. 61/173,782, filed on Apr. 29, 2009.

(51) Int. Cl. A61F 2/24

(52) U.S. Cl. CPC A61F 2/2418; A61F 2/2412; A61F 2/2436; A61F 2210/0004; A61F 2210/0014; A61F 2220/0008; A61F 2220/005; A61F 2220/0066; A61F 2220/0075; A61F 2230/0054; A61F 2230/0069; A61F 2230/0078; A61F 2250/006; A61F 2250/0098; A61F 2310/00023

(57) ABSTRACT

An apparatus for replacing a native mitral valve or a native tricuspid valve includes a radially expandable and compressible securing member having an atrial end portion, a ventricular end portion, and a main body portion extending between the atrial and ventricular end portions. The securing member includes a first attachment member disposed at the atrial end portion and a second attachment member disposed at the ventricular end portion. The first attachment member includes a mesh structure and is configured to contact and anchor against tissue in an atrium upstream of the native mitral or tricuspid valve. The second attachment member is configured to contact and anchor against tissue in a ventricle downstream of the native mitral valve or native tricuspid valve. The apparatus also includes a radially expandable and compressible annular support member disposed within the securing member, and a prosthetic valve disposed within and secured to the support member.

### CROSS REFERENCE TO RELATED APPLICATIONS

This application is a continuation of U.S. patent application Ser. No. 17/067,580, filed Oct. 9, 2020, which is a continuation of U.S. patent application Ser. No. 12/769,593, filed Apr. 28, 2010, now abandoned, which claims the benefit of U.S. Provisional Application No. 61/173,782, filed Apr. 29, 2009, all of which applications are incorporated herein by reference.

### TECHNICAL FIELD

The present invention relates generally to apparatus and methods for treating heart disease, and more particularly to self-expandable apparatus and methods for treating diseased cardiac valves.

### BACKGROUND OF THE INVENTION

There are two atrioventricular (AV) valves in the heart; one on the left side of the heart and one on the right side of the heart. The left side AV valve is the mitral valve and the right side AV valve is the tricuspid valve. Both of these valves are subject damage and dysfunction that requires that the valve be repaired or replaced.

The mitral and tricuspid valves differ significantly in anatomy. While the annulus of the mitral valve is generally D-shaped, the annulus of the tricuspid valve is more circular. The effects of valvular dysfunction vary between the mitral valve and the tricuspid valve. Mitral valve regurgitation has more severe physiological consequences to the patient than does tricuspid valve regurgitation, a small amount of which is tolerable.

In mitral valve insufficiency, the valve leaflets do not fully close and a certain amount of blood leaks back into the left atrium when the left ventricle contracts. As a result, the heart has to work harder by pumping not only the regular volume of blood, but also the extra volume of blood that regurgitated back into the left atrium. The added workload creates an undue strain on the left ventricle. This strain can eventually wear out the heart and result in morbidity. Consequently, proper function of the mitral valve is critical to the pumping efficiency of the heart.

Mitral and tricuspid valve disease is traditionally treated by either surgical repair with an annuloplasty ring or surgical replacement with a valve prosthesis. Surgical valve replacement or repair, however, is often an exacting operation. The operation requires the use of a heart-lung machine for external circulation of the blood as the heart is stopped and then opened during the surgical intervention. Once the heart is opened, the artificial cardiac valves and/or annuloplasty rings are sewed in under direct vision.

Surgical repair of the AV valves exposes patients (i.e., elderly patients) to many risks. A minimally invasive procedure that could be performed under local anesthesia in the cardiac catheterization lab, rather than in cardiac surgery, could therefore offer tremendous benefits to these patients. Consequently, an apparatus for replacing a diseased AV valve using a minimally invasive approach would be very helpful in providing additional opportunities to treat patients with valvular insufficiency and/or end stage heart failure.

### SUMMARY OF THE INVENTION

According to one aspect of the present invention, an apparatus is provided for replacing a native cardiac valve. The native cardiac valve has at least one leaflet and is surrounded by a native cardiac valve annulus. The native cardiac valve annulus has a superior aspect and an inferior aspect. The apparatus comprises a barbell-shaped, expandable anchoring member including a first end portion, a second end portion, and a main body portion extending between the end portions. The main body portion includes a channel defined by an inner surface and an outer surface. Each of the first and second end portions has a diameter greater than the diameter of the main body portion. The first and second end portions are sized to respectively contact the superior and inferior aspects of the native cardiac valve annulus when the expandable anchoring member is in an expanded configuration. The apparatus also includes an expandable support member operably disposed within the main body portion of the expandable anchoring member, and a prosthetic cardiac valve secured within the expandable support member.

According to another aspect of the present invention, a method is provided for replacing a native cardiac valve. The native cardiac valve has at least one leaflet and is surrounded by a native cardiac valve annulus. The native cardiac valve annulus has a superior aspect and an inferior aspect. One step of the method comprises providing an apparatus including a barbell-shaped expandable anchoring member, an expandable support member, and a prosthetic cardiac valve secured within the expandable support member. The expandable support member is secured within the expandable anchoring member. Each of the first and second end portions has a diameter greater than the diameter of the main body portion. The apparatus is placed into a delivery catheter, and the delivery catheter inserted into an atrial chamber. The delivery catheter is advanced until the delivery catheter is positioned within the native cardiac valve annulus. Next, the apparatus is removed from the delivery catheter so that the expandable anchoring member obtains an expanded configuration and the first and second end portions of the expandable anchoring member respectively contact the superior and inferior aspects of the native cardiac valve annulus and thereby secure the expandable anchoring member in the native cardiac annulus.

According to another aspect of the present invention, an apparatus is provided for replacing a native cardiac valve. The native cardiac valve has at least one leaflet and is surrounded by a native cardiac valve annulus. The native cardiac valve annulus has a superior aspect and an inferior aspect. The apparatus comprises an expandable support member, a prosthetic cardiac valve operably secured within the expandable support member, and a securing member operably connected to the expandable support member. The securing member comprises an elongated body member having a first end, a second end, and a main body portion extending between the first and second ends. The second end includes a first attachment member operably connected thereto for contacting the inferior aspect of the native cardiac valve annulus when the expandable support member is in an expanded configuration.

According to another aspect of the present invention, a method is provided for replacing a native cardiac valve. The native cardiac valve has at least one leaflet and is surrounded by a native cardiac valve annulus. The native cardiac valve annulus has a superior aspect and an inferior aspect. One step of the method comprises providing an apparatus including an expandable support member having a prosthetic cardiac valve secured therein and a securing member operably connected to the expandable support member. The securing member comprises an elongated body member having a first end, a second end, and a main body portion extending between the first and second ends. The second end includes a first attachment member operably connected thereto. The expandable anchoring member is placed into a delivery catheter, and the delivery catheter is then inserted into an atrial chamber. The delivery catheter is advanced until the delivery catheter is positioned within the native cardiac valve annulus. Next, the apparatus is removed from the delivery catheter so that the expandable support member obtains an expanded configuration and the first attachment member of the securing member contacts the inferior aspect of the native cardiac valve annulus and thereby secures the expandable support member in the native cardiac valve annulus.

According to another aspect of the present invention, an apparatus is provided for replacing a native cardiac valve. The native cardiac valve has at least one leaflet and is surrounded by a native cardiac valve annulus. The native cardiac valve annulus has a superior aspect and an inferior aspect. The apparatus comprises a securing member including an elongated body member having a first end, a second end, and a main body portion extending between the first and second ends. The second end includes a first attachment member operably connected thereto for contacting the inferior aspect of the native cardiac valve annulus when the expandable support member is in an expanded configuration. The apparatus also comprises a prosthetic valve operably secured to the securing member.

According to another aspect of the present invention, a method is provided for replacing a native cardiac valve. The native cardiac valve has at least one leaflet and is surrounded by a native cardiac valve annulus. The native cardiac valve annulus has a superior aspect and an inferior aspect. One step of the method includes providing an apparatus comprising a securing member and a prosthetic cardiac valve operably connected to the securing member. The securing member comprises an elongated body member having a first end, a second end, and a main body portion extending between the first and second ends. The second end includes a first attachment member operably connected thereto for contacting the inferior aspect of the native cardiac valve annulus when the expandable support member is in an expanded configuration. The apparatus is placed into a delivery catheter, and the delivery catheter is then inserted into an atrial chamber. The delivery catheter is advanced until the delivery catheter is positioned within the native cardiac valve annulus. Next, the apparatus is removed from the delivery catheter so that the prosthetic cardiac valve expands in place of the native cardiac valve and the first attachment member of the securing member contacts the inferior aspect of the native cardiac valve annulus and thereby secures the prosthetic cardiac valve in the native cardiac valve annulus.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1A is a perspective view of an expandable anchoring member constructed in accordance with the present invention;

FIG. 1B is a perspective view of a prosthetic valve operably secured to an expandable support member;

FIG. 1C is a perspective view of an apparatus for replacing a native cardiac valve constructed in accordance with the present invention;

FIG. 1D is a cross-sectional view of the apparatus shown in FIG. 1C;

FIG. 1E is a top plan view of the apparatus shown in FIG. 1C;

FIG. 2 is a cross-sectional view of a human heart;

FIG. 3A is a perspective view showing an alternative embodiment of the expandable anchoring member in FIG. 1A;

FIG. 3B is a perspective view showing an alternative embodiment of the apparatus in FIG. 1C;

FIG. 4 is a perspective view showing the apparatus of FIG. 1C being delivered to a native mitral valve;

FIG. 5 is a cross-sectional view showing the apparatus of FIG. 1C implanted in a native mitral valve;

FIG. 6A is a cross-sectional view showing another alternative embodiment of the apparatus in FIG. 1C;

FIG. 6B is a cross-sectional view showing an alternative embodiment of the apparatus in FIG. 6A;

FIG. 6C is a cross-sectional view showing another alternative embodiment of the apparatus in FIG. 6A;

FIG. 6D is a cross-sectional view showing another alternative embodiment of the apparatus in FIG. 6A;

FIG. 6E is a cross-sectional view showing another alternative embodiment of the apparatus in FIG. 6A;

FIG. 7 is a perspective view showing the apparatus of FIG. 6A being delivered to a native mitral valve;

FIG. 8 is a cross-sectional view showing the apparatus of FIG. 6A implanted in a native mitral valve;

FIG. 9 is a cross-sectional view showing another alternative embodiment of the apparatus in FIG. 6A;

FIG. 10 is a cross-sectional view showing the apparatus of FIG. 9 implanted in a native mitral valve;

FIG. 11 is a cross-sectional view showing another alternative embodiment of the apparatus in FIG. 6A; and

FIG. 12 is a cross-sectional view showing the apparatus of FIG. 11 implanted in a native mitral valve.

### DETAILED DESCRIPTION

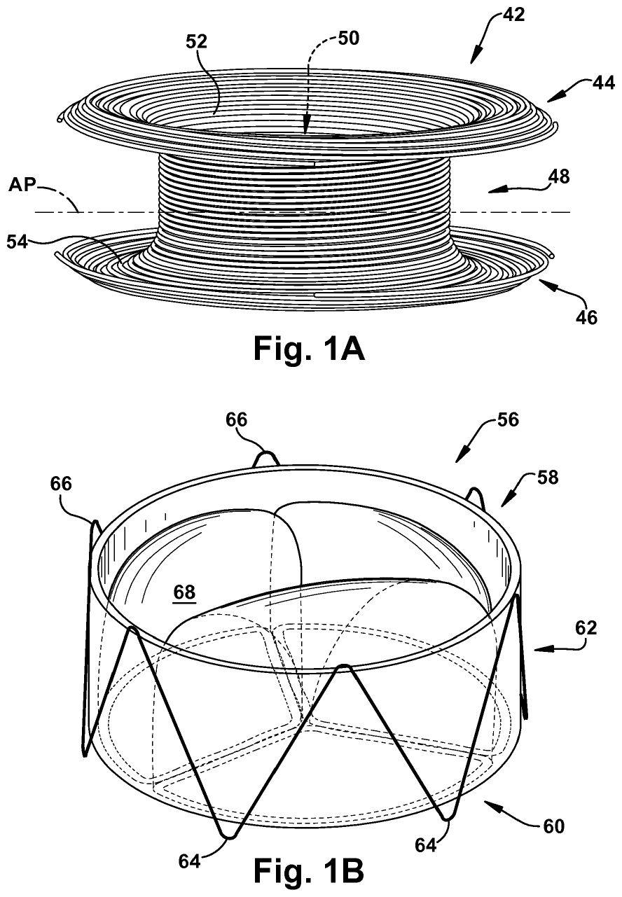

The present invention relates generally to apparatus and methods for treating heart disease, and more particularly to self-expandable apparatus and methods for treating diseased cardiac valves. As representative of the present invention, FIGS. 1A-E illustrate one embodiment of an apparatus 10 for replacing a native cardiac valve. Although the present invention is described herein as being useful for treating a diseased mitral valve, it should be appreciated that other cardiac valves, such as the tricuspid valve, the pulmonary valve, and the aortic valve are also treatable according to the present invention.

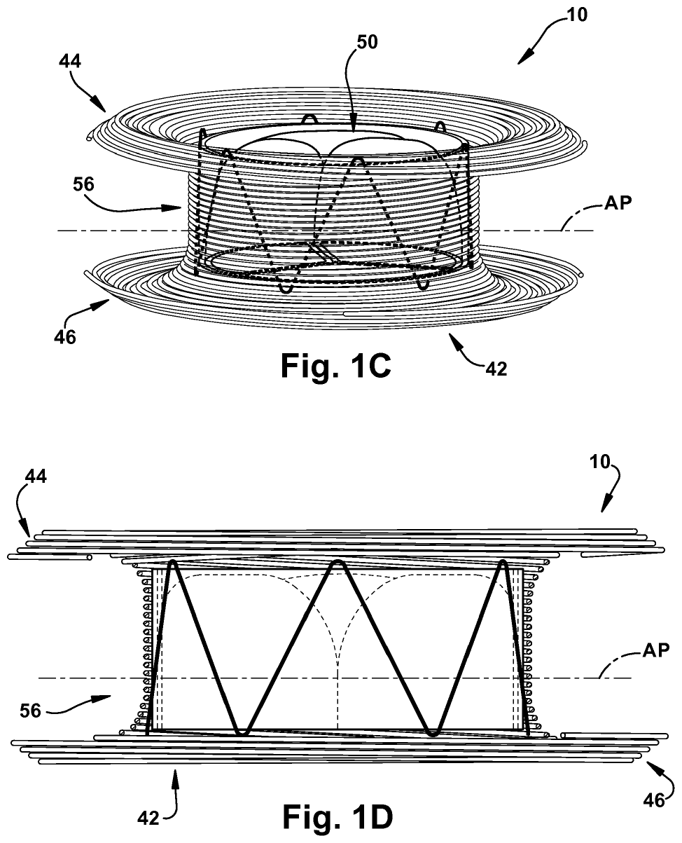

FIG. 2 shows a human heart 12. The human heart 12 contains four chambers: the right and left atria 14 and 16 and the right and left ventricles 18 and 20. The thin-walled right atrium 14 receives deoxygenated blood from the superior vena cava 22, the inferior vena cava (not shown), and from the coronary sinus (not shown). The thin-walled left atrium 16 receives oxygenated blood from pulmonary veins 24. The right and left ventricles 18 and 20 pump oxygenated and deoxygenated blood, respectively, throughout the body, and the pocket-like pulmonary (not shown) and aortic 26 semilunar valves prevent reflux into the ventricles.

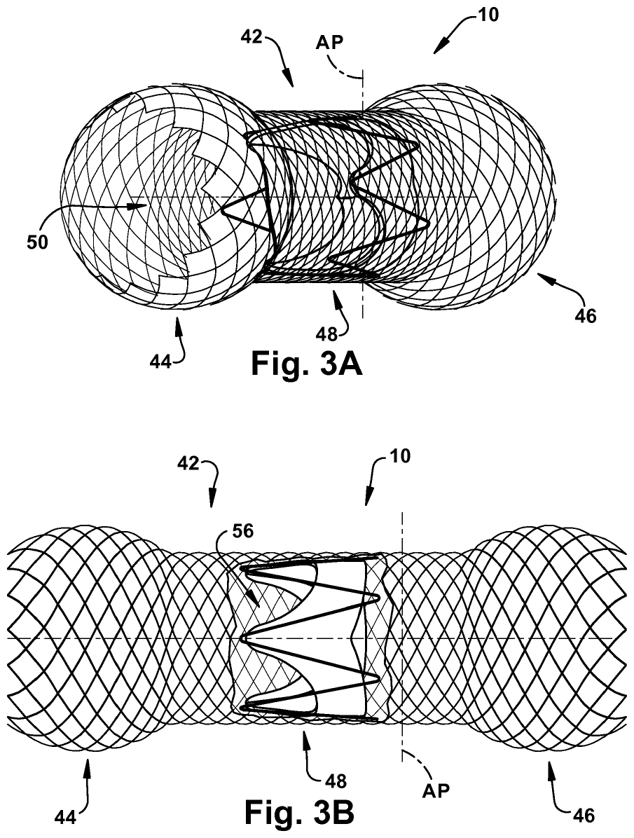

Atrial blood is pumped through the atrioventricular orifices, guarded by the 3-cusp tricuspid valve 28 on the right and the 2-cusp mitral valve 30 on the left. The mitral valve 30 is formed by two leaflets; namely, the anterior leaflet 32 and the posterior leaflet 34. The anterior leaflet 32 extends along a generally planar base of a D-shaped mitral annulus 36 (FIG. 5), while the posterior leaflet 34 (FIG. 2) extends arcuately around the curved portion of the annulus. The mitral and tricuspid valves 28 and 30 are secured to the papillary muscles 38 in the right and left ventricles 18 and 20 by tendinous chordae tendineae 40, and by the mitral annulus 36 and the tricuspid annulus (not shown in detail).

Referring again to FIGS. 1A-E, one embodiment of the present invention includes an apparatus 10 for replacing a native cardiac valve, such as a native mitral valve 30. As shown in FIG. 1A, the apparatus 10 comprises a barbell-shaped expandable anchoring member 42. The expandable anchoring member 42 includes a first end portion 44, a second end portion 46, and a main body portion 48 extending between the first and second end portions. The main body portion 48 includes a channel 50 defined by an inner surface 52 and an outer surface 54. The main body portion 48 has a generally cylindrical shape and is adapted to conform to the three-dimensional shape of a native cardiac valve annulus. It will be appreciated that the size and shape of the main body portion 48 may be varied as needed. For example, the diameter, circumference, and/or length of the main body portion 48 may be varied so that the expandable anchoring member 42 more readily conforms to the shape of a native cardiac valve annulus.

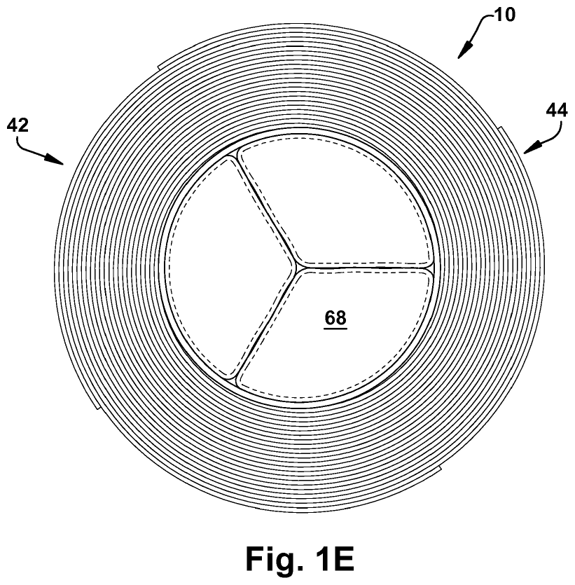

Each of the first and second end portions 44 and 46 of the expandable anchoring member 42 has a diameter that is greater than the diameter of the main body portion 48. As described in more detail below, the first and second end portions 44 and 46 are sized to respectively contact the superior and inferior aspects of a native cardiac valve annulus when the expandable anchoring member 42 is in an expanded configuration. The first and second end portions 44 and 46 can have identical or different configurations. As shown in FIG. 1A, for example, the first and second end portions 44 and 46 have a hemi-spherical shape with respect to an axial plane AP of the main body portion 48. Alternatively, the first and second end portions 44 and 46 of the expandable anchoring member 42 can have bulbous shape (FIGS. 3A-B).

The expandable anchoring member 42 is comprised of a single strand of a flexibly resilient material, such as Nitinol, stainless steel, or other suitable medical grade metals or plastics having shape memory characteristics. It will be appreciated, however, that the expandable anchoring member 42 can alternatively be comprised of multiple strands. Additionally, at least a portion of the expandable anchoring member 42 may be made from a bioabsorbable material including, for example, magnesium alloy, dendrimers, biopolymers such as thermoplastic starch, polyalctides, cellulose, and aliphatic aromatic copolyesters. The expandable anchoring member 42 may also be made of a radio-opaque material or include radio-opaque markers (not shown) to facilitate fluoroscopic visualization. The flexible and expandable properties of the expandable anchoring member 42 facilitate delivery of the apparatus 10 to a diseased native cardiac valve.

The apparatus 10 (FIG. 1C) additionally includes an expandable support member 56 (FIG. 1B) operably disposed within the main body portion 48 of the expandable anchoring member 42. The expandable support member 56 can be secured within the main body portion 48 using any one or combination of known fastening means (not shown), such as sutures, clips, pins, staples, adhesives, or the like. As shown in FIG. 1B, the expandable support member 56 includes oppositely disposed proximal and distal end portions 58 and 60, and a main body portion 62 extending between the end portions. The expandable support member 56 is both flexible and resilient and, as discussed in more detail below, can be made of a shape memory material such as Nitinol, stainless steel, or other suitable medical grade metals or plastics having shape memory characteristics.

The expandable support member 56 may additionally or optionally be made from a bioabsorbable material including, for example, magnesium alloy, dendrimers, biopolymers such as thermoplastic starch, polyalctides, cellulose, and aliphatic aromatic copolyesters. The expandable support member 56 may also be made of a radio-opaque material or include radio-opaque markers to facilitate fluoroscopic visualization. The flexible and expandable properties of the expandable support member 56 facilitate placement and movement of the expandable support member within the main body portion 48 of the expandable anchoring member 42.

The expandable support member 56 comprises a continuous series of W-shaped segments which collectively form a mesh-like configuration. It is contemplated, however, that other geometries may be used. The lower tips 64, as viewed in FIG. 1B, of the W-shaped segments form the distal end portion 60 of the expandable support member 56, and the upper tips 66 of the W-shaped segments form the proximal end portion 58 of the expandable support member. Other examples of expandable support members 56 which may be used as part of the present invention are disclosed in U.S. Patent Pub. No. 2007/0255389 A1, the entirety of which is hereby incorporated by reference.

As shown in FIGS. 1B-E, the expandable support member 56 also includes a prosthetic valve 68 operably secured therein. The prosthetic valve 68 is secured to the expandable support member 56 using any one or combination of known fastening means (not shown), such as sutures, pins, clips, staples, adhesives, or the like. Examples of prosthetic valves 68 are known in the art and can include, for instance, the prosthetic valves disclosed in U.S. Pat. No. 5,156,621, which is hereby incorporated by reference in its entirety.

The prosthetic valve 68 may be fixed and preserved using a variety of known methods. The use of chemical processes for the fixation and preservation of biological tissues have been described and are readily available in the art. For example, glutaraldehyde and other related aldehydes have seen widespread use in preparing cross-linked biological tissues. Glutaraldehyde is a five carbon aliphatic molecule with an aldehyde at each end of the chain, rendering it bifunctional. These aldehyde groups react under physiological conditions with primary amine groups on collagen molecules resulting in the cross-linking of collagen containing tissues. Methods for glutaraldehyde fixation of biological tissues have been extensively described and are well known in the art. In general, a biological tissue sample to be cross-linked is simply contacted with a glutaraldeyde solution for a duration effective to cause the desired degree of cross-linking within the biological tissue being treated.

Many variations and conditions have been applied to optimize glutaraldehyde fixation procedures. For example, lower concentrations have been found to be better in bulk tissue cross-linking compared to higher concentrations. It has been proposed that higher concentrations of glutaraldehyde may promote rapid surface cross-linking of the tissue, generating a barrier that impedes or prevents the further diffusion of glutaraldehdye into the tissue bulk. For most bioprosthesis applications, however, the tissue is treated with a relatively low concentration glutaraldehyde solution, e.g., typically between 0.1%-5%, for 24 hours or more to ensure optimum fixation. Various other combinations of glutaraldehyde concentrations and treatment times will also be suitable depending on the objectives for a given application. Examples of such other combinations include, but are not limited to, those disclosed in U.S. Pat. Nos. 6,547,827, 6,561,970, and 6,878,168, all of which are hereby incorporated by reference in their entireties.

In addition to bifunctional aldehydes, many other chemical fixation procedures have been described. For example, some methods have employed polyethers, polyepoxy compounds, diisocyanates, and azides. These and other approaches are available to the skilled artisan for treating biological tissues, and are suitable for cross-linking vascular graft tissue according to the present invention.

The prosthetic valve 68 may also be treated and preserved with a dry tissue valve procedure as described in U.S. Pat. No. 6,534,004, the entire contents of which are hereby incorporated by reference. Furthermore, the prosthetic valve 68 may be treated with anti-calcification solutions, such as XenoLogiX® (Edwards Lifesciences, Irvine, Calif.), the SynerGraf® (CryoLife, Inc., Kennesaw, Ga.) treatment process, and/or anti-calcification agents, such as a-amino oleic acid.

The apparatus 10 may further include a layer (not shown) of biocompatible material covering at least a portion of the expandable anchoring member. The layer of biocompatible material may be synthetic, such as Dacron® (Invista, Wichita, Kans.), woven velour, polyurethane, polytetrafluoroethylene (PTFE), expanded PTFE, Gore-Tex® (W. L. Gore & Associates, Flagstaff, Ariz.), or heparin-coated fabric. Alternatively, the layer may be a biological material, such as bovine or equine pericardium, peritoneal tissue, an allograft, a homograft, a patient graft, or a cell-seeded tissue. The layer can cover either the inner surface 52 of the expandable anchoring member 42, the outer surface 54 of the expandable anchoring member, or a combination thereof. The layer may be attached around the entire circumference of the expandable anchoring member 42 or, alternatively, may be attached in pieces or interrupted sections to allow the expandable anchoring member to more easily expand and contract. By covering a portion of the expandable anchoring member 42 with a layer of biocompatible material, the hemocompatibility of the apparatus 10 may be improved.

At least a portion of the apparatus 10 may be treated with a therapeutic agent for eluting into cardiac tissue and/or blood. The therapeutic agent may be capable of treating a variety of pathological conditions including, but not limited to, thrombosis, stenosis and inflammation. Accordingly, the therapeutic agent may include at least one of an anticoagulant, an antioxidant, a fibrinolytic, a steroid, an anti-apoptotic agent, an anti-inflammatory agent, a receptor agonist or antagonist, and/or a hormone.

Optionally or additionally, the therapeutic agent may be capable of treating or preventing other diseases or disease processes, such as microbial infections. In these instances, the therapeutic agent may include an anti-microbial agent and/or a biological agent such as a cell, peptide or nucleic acid. The therapeutic agent can be simply linked to a surface of the apparatus 10, embedded and released from within polymer materials, such as a polymer matrix, or surrounded by and released through a carrier. The entire apparatus 10, or only a portion thereof, may be treated with the therapeutic agent. Additionally, different portions of the apparatus 10 may be treated with different therapeutic agents.

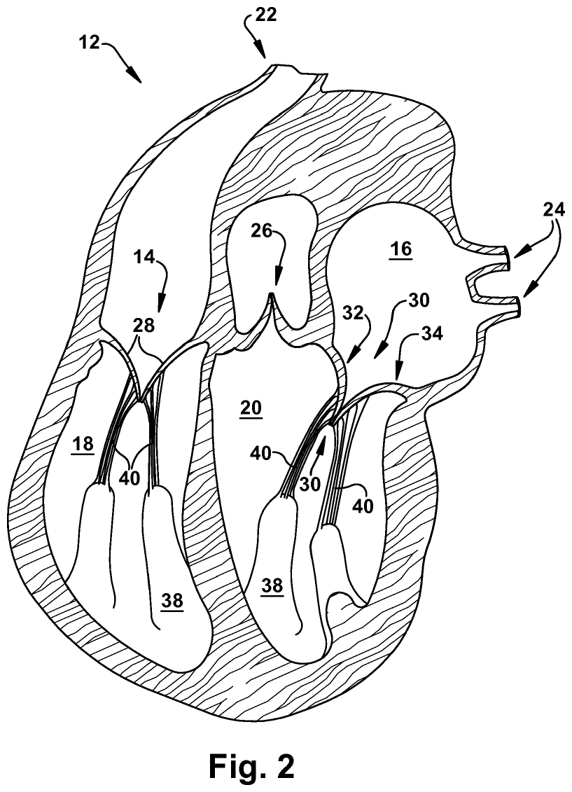

The apparatus 10 shown in FIGS. 1A-E and FIGS. 3A-B can be used to replace a diseased mitral valve 30, for example, using any one or combination of known surgical methods. As shown in FIGS. 4-5, for example, an apical puncture method can be used to respectively deliver the apparatus 10 shown in FIG. 1C. It will be appreciated, however, that other percutaneous, transvascular, and/or open surgical procedures may be used to deliver the apparatus 10 to a diseased cardiac valve. For example, the apparatus 10 can be delivered to the tricuspid valve 28 via the pulmonary artery (not shown) or to the mitral valve 30 via the aortic valve 26. It will also be appreciated that the method of the present invention will typically entail gaining access to a beating heart 12; however, the present invention may also be used for intravascular stopped-heart access as well as stopped-heart open chest procedures.

FIG. 4 illustrates one step of an apical puncture method for delivering the apparatus 10 shown in FIG. 1C to a native cardiac valve, such as the mitral valve 30. One step of the method includes placing the apparatus 10 into a delivery catheter 70. As shown in FIG. 4, the delivery catheter 70 has proximal end portion 72 and a distal end portion 74. The delivery catheter 70 is shaped to facilitate insertion and removal of the apparatus 10 into and out of a puncture tool 76 (not shown in detail). The delivery catheter 70 may be constructed from a rigid, semi-rigid, or flexible material. For example, the delivery catheter 70 may be made of a flexible elastic material, such as a shape memory alloy, a super-elastic material (e.g., Nitinol, spring stainless steel, etc.), or plastic. Alternatively, the delivery catheter 70 may be made of a rigid material, such as hardened plastic, silicon, polyurethane, or the like.

Prior to placing the apparatus 10 into the delivery catheter 70, the dimensions of the native mitral valve 30 and the native mitral annulus 36 are determined. Various methods and devices for determining the dimensions of cardiac valves and cardiac valve annuluses are known in the art and include, for example, echocardiogram, computed tomography, magnetic resonance imaging, fluoroscopy, and angiography. After determining the dimensions of the native mitral valve 30 and the native mitral annulus 36, an appropriately-sized apparatus 10 is chosen for implantation. For example, the main body portion 48 of the expandable anchoring member 42 can be appropriately-sized so that the diameter of the main body portion corresponds to the diameter of the native mitral annulus 36. Additionally, the first and second end portions 44 and 46 of the expandable anchoring member 42 can also be appropriately-sized so that the first and second end portions respectively contact the superior and inferior aspects 78 and 80 of the mitral annulus 36 when the expandable anchoring member is in an expanded configuration.

After selecting an appropriately-sized apparatus 10ₐ the puncture tool 76 is used to puncture the chest wall and extend through the apical portion of the left ventricle 20 into the left ventricular chamber. The delivery catheter 70 is then urged through the puncture tool 76 as shown in FIG. 4 so that the delivery catheter is positioned at a distal end portion 82 of the puncture tool. Both the puncture tool 76 and the delivery catheter 70 are then progressively withdrawn so that the first end portion 44 of the expandable anchoring member 42 expands to contact the superior aspect 78 of the native mitral annulus 36. Once the delivery catheter 70 and the puncture tool 76 have been completely withdrawn from the left ventricle 20, the main body portion 48 and the second end portion 46 of the expandable anchoring member 42 expand into the native mitral annulus 36 (FIG. 5). With the apparatus 10 securely positioned in the native mitral annulus 36, normal blood flow can resume through the prosthetic valve 68.

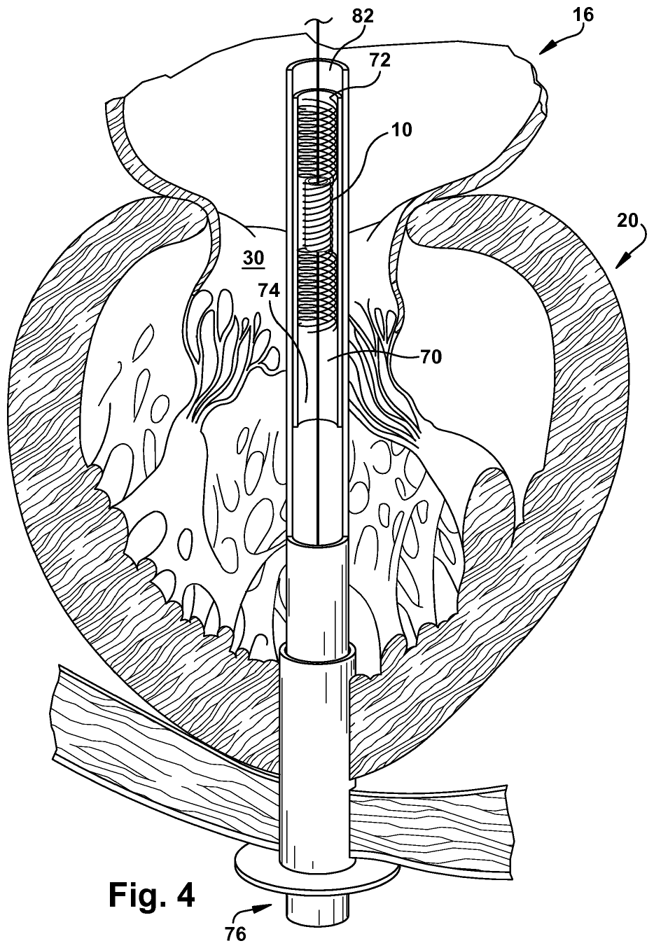

Another embodiment of the present invention is illustrated in FIGS. 6A-E. The apparatus 10ₐ is identically constructed as the apparatus 10 shown in FIGS. 1A-E, except where as described below. In FIGS. 6A-E, structures that are identical as structures in FIGS. 1A-E use the same reference numbers, whereas structures that are similar but not identical carry the suffix “a”.

An apparatus 10ₐ for replacing a native cardiac valve, such as a native mitral valve 30 can comprise an expandable support member 56 and a prosthetic cardiac 68 valve operably secured within the expandable support member. As shown in FIGS. 6A-E, the apparatus 10ₐ can further include a securing member 84 operably connected to the expandable support member 56. The securing member 84 can comprise an elongated body member 86 having a first end 88, a second end 90, and a main body portion 92 extending between the first and second ends. The securing member 84 can be operably secured to the expandable support member 56 using any one or combination of known fastening means (not shown), such as sutures, clips, pins, staples, adhesives, or the like.

The second end 90 of the elongated body member 84 can include a first attachment member 94 operably connected thereto for contacting the inferior aspect of the native cardiac valve annulus when the expandable support member 56 is in an expanded configuration. As shown in FIGS. 6A-E, the first end 88 of the elongated body member 86 can also include a second attachment member 96 operably connected thereto for contacting the superior aspect of the native cardiac valve annulus when the expandable support member 56 is in an expanded configuration. The first and second attachment members 94 and 96 can be made from any one or combination of flexibly resilient, medical grade materials, including, for example, Nitinol, stainless steel, or other suitable metals or plastics having shape memory characteristics.

The first and second attachment members 94 and 96 can have a variety of configurations. As shown in FIGS. 6A-C, for example, the first and second attachment members 94 and 96 can include flexible, rod-shaped members 98. The rod-shaped members 98 can be joined to or integrally formed with the elongated body member 86 so that the rod-shaped members can transition from a collapsed configuration (indicated by the dashed lines) to an expanded configuration. In the collapsed configuration, the rod-shaped members 98 can extend substantially parallel to the elongated body member 86. In the expanded configuration, the rod-shaped members 98 can extend substantially axial to the elongated body member 86. As described in more detail below, the rod-shaped members 98 located at the first and second ends 88 and 90 of the elongated body member 86 can respectively contact the superior and inferior aspects 78 and 80 of the mitral annulus 36 when the apparatus 10ₐ is in an expanded configuration.

As shown in FIGS. 6B-D, the first and second attachment members 94 and 96 can also comprise a windable coil 100. The windable coil 100 can be made of a flexible wire or rod capable of transitioning between an expanded configuration and a collapsed configuration. In the expanded configuration (indicated by the dashed lines), the windable coil 100 can obtain a substantially linear configuration so that the windable coil extends substantially parallel to the elongated body member 86. In the collapsed configuration (FIGS. 6B-D), the windable coil 100 can obtain a substantially circular configuration and extend substantially axial to the elongated body member 86. As described in more detail below, each of the windable coils 100 located at the first and second ends 88 and 90 of the elongated body member 86 can respectively contact the superior and inferior aspects 78 and 80 of a native mitral annulus 36 when the apparatus 10ₐ is in an expanded configuration.

The first and second attachment members 94 and 96 can additionally comprise an anchoring ring 102 (FIG. 6E). The anchoring ring 102 can be similarly or identically constructed as the first and second end portions 44 and 46 of the expandable anchoring member 42 shown in FIGS. 3A-B. In an expanded configuration, the anchoring ring 102 can have a bulbous shape and extend substantially axial to the elongated body member 86. In a collapsed configuration (indicated by dashed lines), the anchoring ring 102 can extend substantially parallel to the elongated body member 86. The anchoring ring 102 can be made of a flexible, mesh-like material having shape memory characteristics.

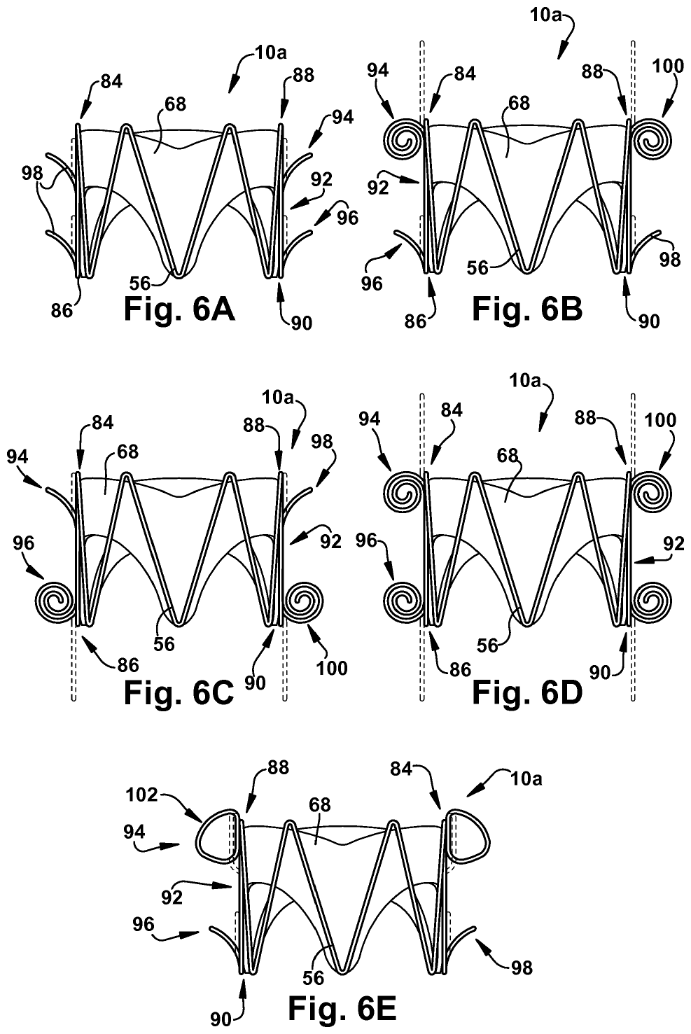

It will be appreciated that the apparatus 10ₐ shown in FIGS. 6A-E can additionally include a layer 104 (FIG. 8) of material that extends around all or a portion of the expandable support member 56. The layer 104 of material can be made of any one or combination of known biocompatible materials, some of which are described above. For example, the layer 104 can be made of PTFE or ePTFE. The layer 104 of material can function as a seal to prevent leakage of blood between the left atrium 16 and the left ventricle 20, for example, when the apparatus 10ₐ is implanted in a heart 12.

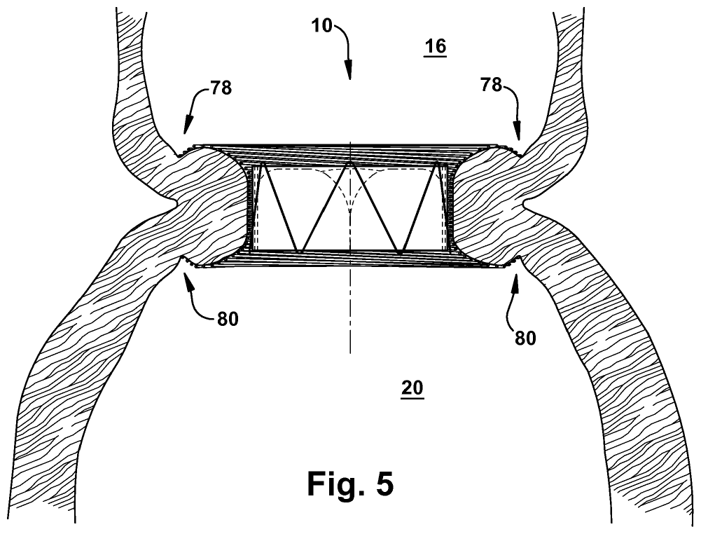

The apparatus 10ₐ shown in FIGS. 6A-E can be used to replace a native mitral valve 30, for example, using any one or combination of known surgical methods. As shown in FIG. 7, for example, an apical puncture method can be used to deliver the apparatus 10ₐ shown in FIG. 6A to a native mitral valve 30. It will be appreciated, however, that other percutaneous, transvascular, and/or open surgical methods may be used to deliver the apparatus 10ₐ to a native mitral valve 30. It will also be appreciated that the method of the present invention will typically entail gaining access to a beating heart 12; however, the present invention may also be used for intravascular stopped-heart access as well as stopped-heart open chest procedures.

The apparatus 10ₐ shown in FIG. 6A can be delivered to a native mitral valve 30 using an apical puncture method similar or identical to the apical puncture method described above. Briefly, one step of the method can include placing the apparatus 10ₐ into a delivery catheter 70. Prior to placing the apparatus 10ₐ into the delivery catheter 70, the dimensions of the native mitral valve 30 and the native mitral annulus 36 can be determined. After selecting an apparatus 10ₐ whose dimensions correspond to the dimensions of the native mitral valve 30 and the native mitral annulus 36, a puncture tool 76 can be used to puncture the chest wall. The puncture tool 76 can then be extended through the apical portion of the left ventricle 20 into the left ventricular chamber. Next, the delivery catheter 70 can be urged through the puncture tool 76 as shown in FIG. 7 so that the delivery catheter is positioned at a distal end portion 82 of the puncture tool.

Both the puncture tool 76 and the delivery catheter 70 can then be progressively withdrawn from the left ventricle 20 so that the expandable support member 56 can expand into contact with the native mitral annulus 36, and the rod-shaped members 98 can transition from the collapsed configuration to the expanded configuration. As the delivery catheter 70 and the puncture tool 76 are completely removed from the left ventricle 20, the rod-shaped members 98 located at the first and second ends 88 and 90 of the elongated body members 86 can respectively contact the superior and inferior aspects 78 and 80 of the mitral annulus 36 and thereby secure the apparatus 10ₐ in the native mitral annulus (FIG. 8). With the apparatus 10ₐ securely positioned in the native mitral annulus 36, normal blood flow can resume through the prosthetic valve 68.

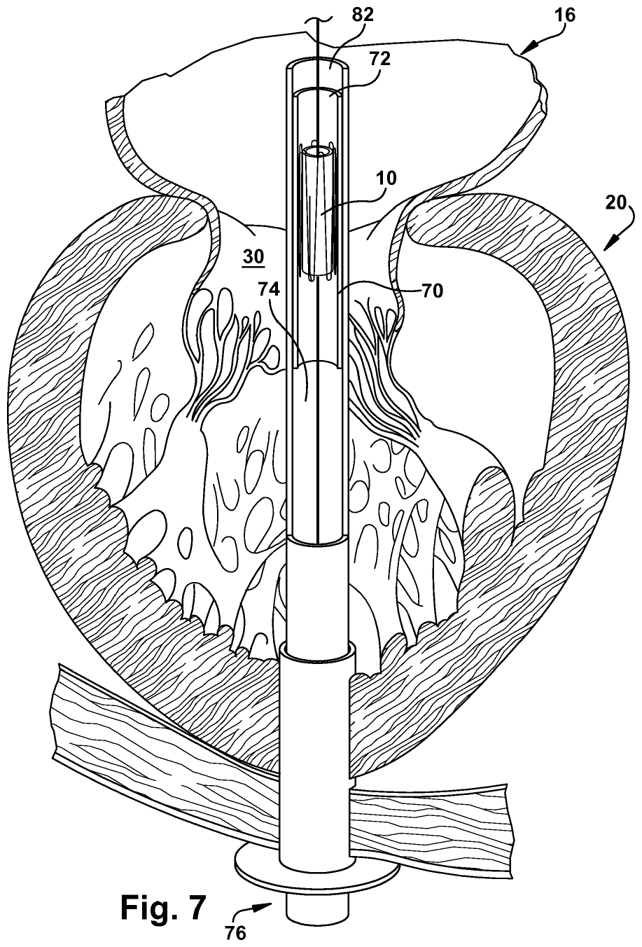

Another embodiment of the present invention is illustrated in FIG. 9. The apparatus 10_{b} is identically constructed as the apparatus 10ₐ shown in FIGS. 6A-E, except where as described below. In FIG. 9, structures that are identical as structures in FIGS. 6A-E use the same reference numbers, whereas structures that are similar but not identical carry the suffix “b”.

An apparatus 10_{b} for replacing a native cardiac valve, such as a mitral valve 30 can comprise an expandable support member 56 and a prosthetic valve 68 operably secured within the expandable support member. As shown in FIG. 9, the apparatus 10_{b} can further include a securing member 84 operably connected to the expandable support member 56. The securing member 84 can comprise an elongated body member 86 having a first end 88, a second end 90, and a main body portion 92 extending between the first and second ends. The securing member 84 can be operably secured to the expandable support member 56 using any one or combination of known fastening means (not shown), such as sutures, clips, pins, staples, adhesives, or the like.

The second end 90 of the elongated body member 86 can also include a first attachment member 94 operably connected thereto for embedding into the annular tissue at the inferior aspect of a native cardiac valve annulus. As shown in FIG. 9, the first attachment member 94 can include at least one rod-shaped puncturing member 106. The puncturing member 106 can have a fixed length or, alternatively, the puncturing member can have a compressible, spring-like configuration (not shown). The puncturing member 106 can have a needle- or barb-like shape to facilitate penetration of the puncturing member into annular tissue.

The apparatus 10_{b} is capable of transitioning between a collapsed configuration and an expanded configuration. As shown in FIG. 9, the puncturing member 106 can extend substantially axial to the elongated body member 86 in the expanded configuration. In the collapsed configuration (indicated by the dashed lines), the puncturing member 106 can extend substantially parallel to the elongated body member 86. As described in more detail below, the puncturing member 106 can be used to secure the apparatus 10_{b} in a native cardiac valve annulus.

The apparatus 10_{b} shown in FIG. 9 can be delivered to a native cardiac valve, such as a mitral valve 30 using an apical puncture method similar or identical to the apical puncture method described above. Briefly, one step of the method can include placing the apparatus 10_{b} into a delivery catheter 70. Prior to placing the apparatus 10_{b} into the delivery catheter 70, the dimensions of the native mitral valve 30 and the native mitral annulus 36 can be determined. After selecting an apparatus 10_{b} whose dimensions correspond to the dimensions of the native mitral valve 30 and the native mitral annulus 36, the puncture tool 76 can be used to puncture the chest wall. The puncture tool 76 can then be extended through the apical portion of the left ventricle 20 into the left ventricular chamber. Next, the delivery catheter 70 can be urged through the puncture tool 76 so that the delivery catheter is positioned at a distal end portion 82 of the puncture tool (not shown).

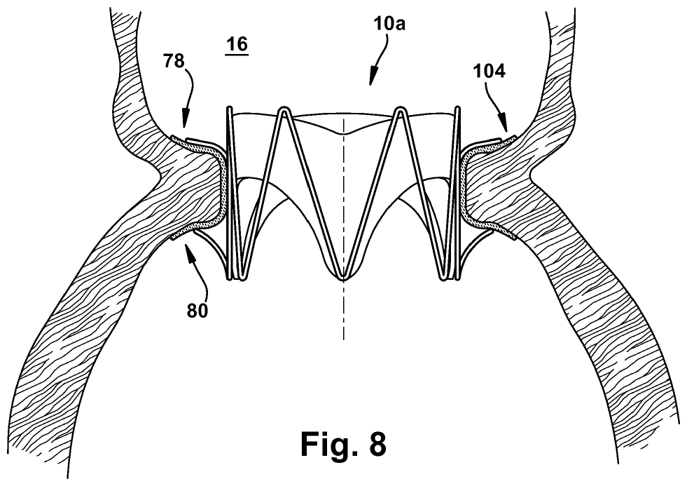

Both the puncture tool 76 and the delivery catheter 70 can then be progressively withdrawn from the left ventricle 20 so that the expandable support member 56 expands into contact with the native mitral annulus 36. As the delivery catheter 70 and the puncture tool 76 are completely removed from the left ventricle 20, each of the puncture members 106 can transition from the collapsed configuration to the expanded configuration. In the expanded configuration, each of the puncture members 106 can penetrate into the annular tissue at the inferior aspect 80 of the native mitral annulus 36 and thereby secure the apparatus 10_{b} in the native mitral annulus (FIG. 10). With the apparatus 10_{b} securely positioned in the native mitral annulus 36, normal blood flow can resume through the prosthetic valve 68.

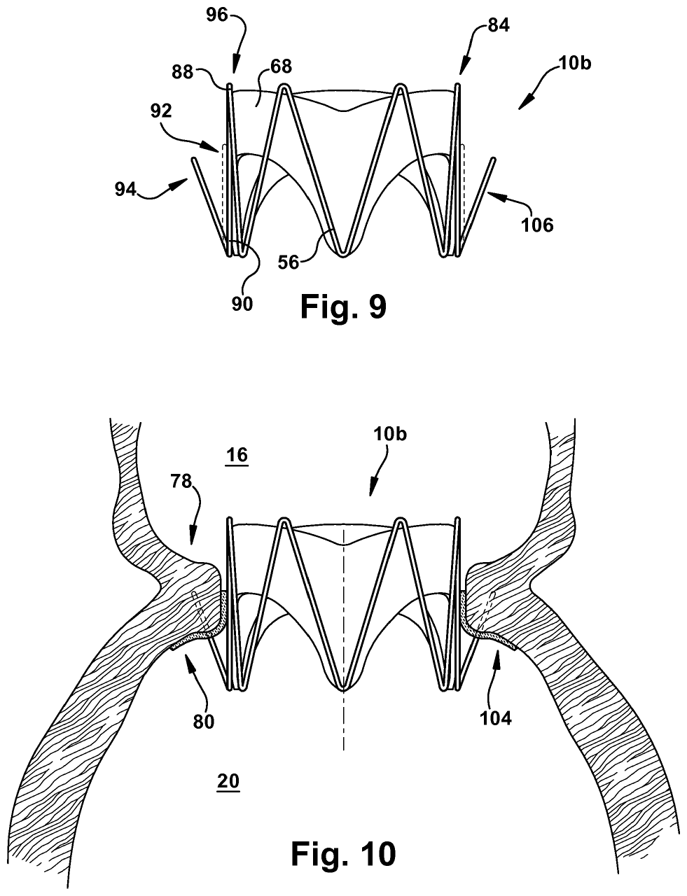

Another embodiment of the present invention is illustrated in FIG. 11. The apparatus 10_{c} is identically constructed as the apparatus 10ₐ shown in FIGS. 6A-E, except where as described below. In FIG. 11, structures that are identical as structures in FIGS. 6A-E use the same reference numbers, whereas structures that are similar but not identical carry the suffix “c”.

An apparatus 10_{c} for replacing a native cardiac valve, such as a mitral valve 30 can comprise a prosthetic valve 68 operably secured to a securing member 84. The securing member 84 can comprise an elongated body member 86 having a first end 88, a second end 90, and a main body portion 92 extending between the first and second ends. The securing member 84 can be operably secured to the prosthetic valve 68 using any one or combination of known fastening means (not shown), such as sutures, clips, pins, staples, adhesives, or the like.

The second end 90 of the elongated body member 86 can include a first attachment member 94 operably connected thereto for contacting the inferior aspect of a native cardiac valve annulus when the apparatus 10_{c} is in an expanded configuration. As shown in FIG. 11, the first end 88 of the elongated body member 86 can also include a second attachment member 96 operably connected thereto for contacting the superior aspect of a native cardiac valve annulus when the apparatus 10_{c} is in an expanded configuration. The first and second attachment members 94 and 96 can be made from any one or combination of flexibly resilient, medical grade materials, including, for example, Nitinol, stainless steel, or other suitable metals or plastics having shape memory characteristics.

The first and second attachment members 94 and 96 can have a variety of configurations. As shown in FIG. 11, for example, the first and second attachment members 94 and 96 can include flexible, rod-shaped members 98. It will be appreciated, however, that the first and second attachment members 94 and 96 can also comprise any one or combination of the structures illustrated in FIGS. 6A-E. The rod-shaped members 98 can be joined to or integrally formed with the elongated body member 86 so that the rod-shaped members can transition from a collapsed configuration (indicated by dashed lines) to an expanded configuration. In the collapsed configuration, the rod-shaped members 98 can extend substantially parallel to the elongated body member 86. In the expanded configuration, the rod-shaped members 98 can extend substantially axial to the elongated body member 86. As described in more detail below, the rod-shaped members 98 located at the first and second ends 88 and 90 of the elongated body member 86 can respectively contact the superior and inferior aspects 78 and 80 of the mitral annulus 36 when the apparatus 10_{c} is in an expanded configuration.

The apparatus 10_{c} shown in FIG. 11 can be used to replace a native cardiac valve, such as a native mitral valve 30 using an apical puncture method similar or identical to the apical puncture method described above. Briefly, one step of the method can include placing the apparatus 10_{c} into a delivery catheter 70. Prior to placing the apparatus 10_{c} into the delivery catheter 70, the dimensions of the native mitral valve 30 and the native mitral annulus 36 can be determined. After selecting an apparatus 10_{c} whose dimensions correspond to the dimensions of the native mitral valve 30 and the native mitral annulus 36, a puncture tool 76 can be used to puncture the chest wall. The puncture tool 76 can then be extended through the apical portion of the left ventricle 20 into the left ventricular chamber. Next, the delivery catheter 70 can be urged through the puncture tool 76 so that the delivery catheter is positioned at a distal end portion 82 of the puncture tool (not shown).

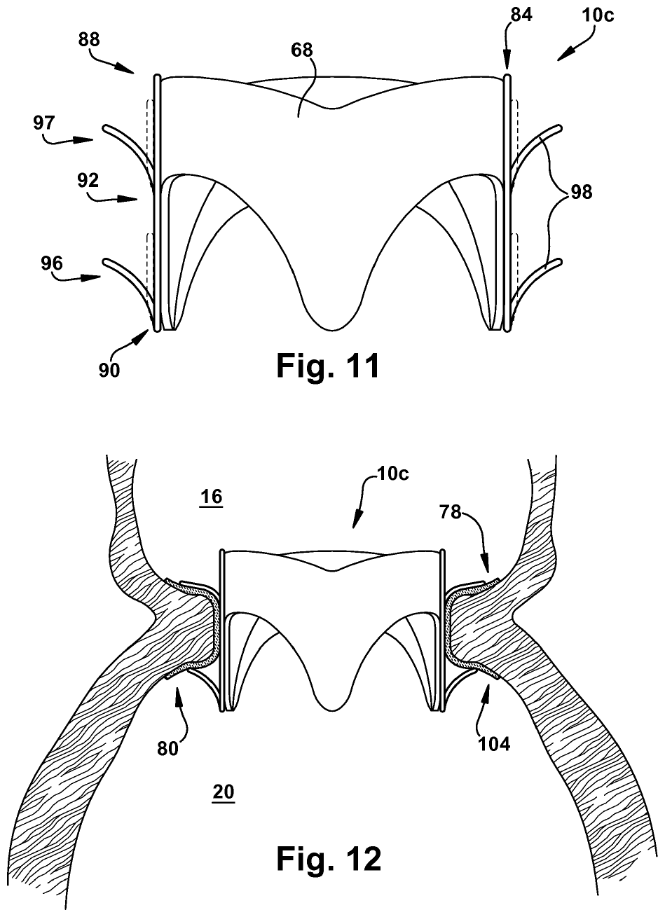

Both the puncture tool 76 and the delivery catheter 70 can then be progressively withdrawn from the left ventricle 20 so that prosthetic valve 68 expands into contact with the native mitral annulus 36, and the rod-shaped members 98 located at the first end 88 of each of the elongated body members 86 transitions from the collapsed configuration to the expanded configuration. As the delivery catheter 70 and the puncture tool 76 are completely removed from the left ventricle 20, the rod-shaped members 98 located at the first and second ends 88 and 90 of each of the elongated body members 86 can respectively contact the superior and inferior aspects 78 and 80 of the mitral annulus 36, thereby securing the apparatus 10_{c} in the native mitral annulus (FIG. 12). With the apparatus 10_{c} securely positioned in the native mitral annulus 36, normal blood flow can resume through the prosthetic valve 68.

From the above description of the invention, those skilled in the art will perceive improvements, changes and modifications. For example, it will be appreciated that the methods of the present invention can include implanting an expandable anchoring member 42 or an expandable support member 56, without a prosthetic valve 68 attached therein, in a native cardiac valve annulus. After the expandable anchoring member 42 or the expandable support member 56 is securely positioned in the native cardiac valve annulus, a prosthetic valve 68 can then be secured therein using any one or combination of known fastening means. Such improvements, changes and modifications are within the skill of the art and are intended to be covered by the appended claims.

We claim:

1. An apparatus for replacing a native mitral valve or a native tricuspid valve, comprising:
a radially expandable and compressible securing member having an atrial end portion, a ventricular end portion, and a main body portion extending between the atrial and ventricular end portions, wherein the securing member comprises a first attachment member disposed at the atrial end portion and a second attachment member disposed at the ventricular end portion, wherein the first attachment member comprises a mesh structure, wherein the first attachment member is configured to contact and anchor against tissue in an atrium upstream of the native mitral valve or native tricuspid valve, wherein the second attachment member is configured to contact and anchor against tissue in a ventricle downstream of the native mitral valve or native tricuspid valve;
a radially expandable and compressible annular support member disposed within the securing member; and
a prosthetic valve disposed within and secured to the support member;
wherein the apparatus is radially compressible to a radially compressed state for placing the apparatus in a delivery catheter and delivering the apparatus to the native mitral valve or the native tricuspid valve and wherein the apparatus can radially expand to a radially expanded state when released from the delivery catheter; and
wherein the securing member and the support member are made of Nitinol and wherein the apparatus can self-expand from the radially compressed state to the radially expanded state when released from the delivery catheter.

2. The apparatus of claim 1, wherein the first and second attachment members are made of Nitinol and wherein the first and second attachment members extend radially outward relative to the support member in the radially expanded state.

3. The apparatus of claim 1, wherein the support member is pre-attached to the securing member with sutures before placing the apparatus in the delivery catheter.

4. The apparatus of claim 1, further comprising a fabric layer attached to and covering at least one of an inner surface and an outer surface of the securing member.

5. The apparatus of claim 1, wherein the prosthetic valve is disposed within a lumen of the support member.

6. The apparatus of claim 1, wherein the securing member comprises a plurality of second attachment members, and wherein each of the second attachment members comprise an elongate shape.

7. An apparatus for replacing a native atrioventricular valve, comprising:
a radially expandable and compressible securing member comprising an inlet end portion, an outlet end portion, a main body portion extending between the inlet end portion and the outlet end portion, and a plurality of attachment members coupled to the outlet end portion, wherein the main body portion is configured to contact an annulus of the native atrioventricular valve when implanted therein, wherein when the apparatus is in the radially expanded state, the attachment members extend radially outwardly from the main body portion and are configured to contact tissue in a ventricle downstream of the native atrioventricular valve, wherein when the apparatus is in the radially expanded state, the inlet end portion of the securing member extends radially outwardly from the main body portion and is configured to contact tissue in an atrium upstream of the native atrioventricular valve, and wherein when the apparatus is in the radially expanded state, the inlet end portion has a greater diameter than the main body portion;
a radially expandable and compressible annular support member disposed within the securing member; and
a prosthetic valve disposed within and secured to the support member;
wherein the apparatus is radially compressible to a radially compressed state for placing the apparatus in a delivery catheter and delivering the apparatus to the native atrioventricular valve and wherein the apparatus can radially expand to a radially expanded state when released from the delivery catheter.

8. The apparatus of claim 7, further comprising a fabric layer attached to the securing member.

9. The apparatus of claim 7, wherein the securing member extends outside of the support member and wherein an outlet end of the support member is positioned adjacent to an outlet end of the securing member.

10. The apparatus of claim 9, wherein an inlet end of the support member is positioned adjacent to an inlet end of the securing member.

11. The apparatus of claim 7, wherein the securing member and the support member are made of Nitinol and wherein the apparatus can self-expand from the radially compressed state to the radially expanded state when released from the delivery catheter.

12. The apparatus of claim 7, wherein the support member is pre-attached to the securing member with sutures before placing the apparatus in the delivery catheter.

13. The apparatus of claim 7, wherein the prosthetic valve is disposed within a lumen of the support member.

14. An apparatus for replacing a native mitral valve or a native tricuspid valve, comprising:
a radially expandable and compressible securing member having a first end portion, a second end portion, and a main body portion extending between the first and second end portions, wherein the securing member comprises a first attachment member disposed at the first end portion and a second attachment member disposed at the second end portion, wherein when the apparatus is in the radially expanded state, the first attachment member extends radially outwardly from the main body portion and is configured to contact and anchor against tissue in an atrium upstream of the native mitral valve or native tricuspid valve, wherein when the apparatus is in the radially expanded state, the second attachment member extends radially outwardly from the main body portion and is configured to contact and anchor against tissue in a ventricle downstream of the native mitral valve or native tricuspid valve;
a radially expandable and compressible annular support member disposed within the securing member, wherein an outlet end of the support member is positioned adjacent to an outlet end of the securing member, wherein the support member is pre-attached to the securing member with sutures before placing the apparatus in a delivery catheter; and
a prosthetic valve disposed within and secured to the support member;
wherein the apparatus is radially compressible to a radially compressed state for placing the apparatus in the delivery catheter and delivering the apparatus to the native mitral valve or the native tricuspid valve and wherein the apparatus can radially expand to a radially expanded state when released from the delivery catheter.

15. The apparatus of claim 14, wherein the first attachment member comprises a mesh structure having shape-memory characteristics.

16. The apparatus of claim 14, wherein the second attachment member comprises an elongate shape and is coupled to the second end portion of the securing member.

17. The apparatus of claim 14, wherein the first and second attachment members are made of Nitinol and wherein the first and second attachment members extend radially outward relative to the support member in the radially expanded state.

18. The apparatus of claim 14, wherein the support member defines a lumen and wherein the prosthetic valve is disposed within the lumen of the support member.

19. The apparatus of claim 14, further comprising a fabric layer attached to the securing member, and wherein the fabric layer at least partially covers an inner surface and/or an outer surface of the securing member.

20. The apparatus of claim 14, wherein an inlet end of the support member is positioned adjacent to an inlet end of the securing member, and wherein an entirety of the support member is disposed within the securing member.
